(19) United States
(12) Patent Application Publication
(10) Pub. No.: US 2003/0139468 A1
(43) Pub. Date: Jul. 24, 2003

(54) Amiodarone solutions suitable for intravenous administration

(75) Inventors: Mahdi B. Fawzi, Morristown, NJ (US); Christian L. Ofslager, Newburgh, NY (US); Yong J. Lee, Monroe, NY (US); Syed M. Shah, East Hanover, NJ (US)

Correspondence Address: WYETH, PATENT LAW GROUP, FIVE GIRALDA FARMS, MADISON, NJ, 07940, US

(73) Assignee: American Home Products Corporation, Madison, NJ

(21) Appl. No.: 10/054,493

(22) Filed: Jan. 22, 2002

(51) Int. Cl. A61K 31/343

(52) U.S. Cl. 514/469

(57) ABSTRACT

Parenteral solutions suitable for intravenous injection are provided comprising amiodarone in an amount of from 0.9 mg/ml to 30 mg/ml, a lactic acid buffer and water, the solution having a pH of 2.5 to 4.5.

### FIELD OF THE INVENTION

The present invention is directed to parenteral formulations of amiodarone suitable for intravenous administration. More specifically, the present invention is directed to parenteral solutions comprising amiodarone and a lactic acid buffer.

### BACKGROUND OF THE INVENTION

2-n-butyl-3-(3,5-diiodo-4-(β-N-diethylaminoethoxy)benzoyl) benzofuran (hereinafter “amiodarone”) is useful as an antiarrhythmic agent. Amiodarone has been approved in the United States for the treatment of life-threatening ventricular tachycardia.

Amiodarone is highly insoluble in water and thus, attempts to develop aqueous formulations suitable for intravenous administration have not always been successful. The commercially available intravenous formulation of amiodarone (Cordarone® IV manufactured and sold by Wyeth-Ayerst Pharmaceuticals) utilizes the surfactant polysorbate 80 and benzyl alcohol to dissolve and solublize the amiodarone. However, the use of polysorbate 80 and other surfactants in intravenous formulations may have undesirable side effects that limit their usefulness. Accordingly, attempts have been made to develop a surfactant-free amiodarone intravenous formulation.

U.S. Pat. No. 5,234,949 discloses aqueous parenteral solutions of amiodarone comprising an acetate buffer having a pH of about 3.5 to 3.8 and a method for treating patients suffering from arrhythmia comprising the intravenous administration of such a solution.

U.S. Pat. No. 6,030,998 discloses compositions suitable for intravenous administration comprising amiodarone dissolved in acetate buffer having a pH of about 3.5 to 4.0 at a concentration of from about 10 mg/ml to 50 mg/ml.

U.S. Pat. No. 6,103,757 discloses a method of treating a human patient suffering from arrhythmia comprising the intravenous administration to the patient of amiodarone dissolved in acetate buffer having a pH of from about 3.5 to 3.8 at a concentration of about 10 mg/ml to 20 mg/ml.

U.S. Pat. No. 6,143,778 discloses a solution suitable for parenteral administration comprising 1.5 to 8 wt. % of amiodarone, a physiologically acceptable buffer solution capable of maintaining the pH of the solution at 2.4 to 3.8 and a non-ionic hydrophilic surfactant. The disclosed buffers include acetate or phosphate buffers.

### SUMMARY OF THE INVENTION

The present invention is directed to parenteral solutions suitable for intravenous administration comprising amiodarone in an amount of from about 0.9 mg/ml to about 30 mg/ml, a lactic acid buffer and water, the solution having a pH of from about 2.5 to 4.5.

### DETAILED DESCRIPTION OF THE INVENTION

The parenteral solutions of the present invention comprise amiodarone in an amount of from about 0.9 mg/ml to about 30 mg/ml, preferably about 0.9 mg/ml to about 15 mg/ml and most preferably about 15 mg/ml.

The lactic acid buffer is used in a concentration of from about 0.05 to about 0.1M and preferably about 0.1 M. Commercially available lactic acid buffers suitable for use in the present invention may be obtained from Sigma-Aldrich.

The parenteral solutions of the present invention have a pH of from about 2.5 to about 4.5 and preferably about 3.5 to about 3.8.

The original solution may be diluted to a concentration of about 0.5 mg/ml to 15 mg/ml with the addition of 5% dextrose to the original formulation.

The parenteral solutions of the present invention may be prepared by any suitable means which would be evident to one skilled in the art in light of the present disclosure. However, it is preferred that the present solutions be prepared in accordance with the following procedure. First, an appropriate amount of lactic acid buffer (e.g., 0.1M) is added to water to form a solution. The pH of this solution is adjusted to 2.5 to 4.5 with a suitable pH adjuster, such as sodium hydroxide. Amiodarone is then added to this lactic acid/water solution and the resultant solution is mixed and heated to a temperature of about 55 to about 65° C. preferably about 60 to about 65° C. Once the amiodarone goes into solution, the resulting solution is cooled to room temperature and placed into suitable sized ampoules. If necessary, the resulting material may be filtered through an appropriate membrane filter. Once placed into ampoules, the solutions may be heated to a temperature of about 62 to about 68° C. and preferably about 65° C. for about 30 minutes. The solution is then quickly cooled to room temperature

In a further embodiment, the present invention relates to lyophilized formulations of amiodarone which, upon reconstitution with water, are suitable for intravenous administration. The lyophilized formulations are in a ‘cake’ form and are prepared by lyophilizing a concentrated amiodarone formulation. The lyophilized formulations comprise amiodarone in an amount of from about 10 mg/ml to about 50 mg/ml, a lactic acid buffer and lactose. Preferably, these formulations may comprise amiodarone in an amount of from about 10 mg/ml to about 20 mg/ml and most preferably about 15 mg/ml. The lactic acid buffer is used in the concentrations set forth above. The formulation may suitably contain about 0.3 g of lactose per lyophilized cake. Suitable adjuvants, such as simethicone, may be added to the lyophilized formulations of the present invention. The pH of the lyophilized formulation, after reconstitution, is in the range of about 2.0 to 4.5.

The lyophilized formulations may be prepared according to the following procedure. A suitable amount of lactose is placed into a container capable of heating. The appropriate amount of amiodarone is then added to the lactose. Water for injection USP is added to the amiodarone/lactose mixture to form a dispersion. An appropriate quantity of lactic acid is then added to the dispersion. While under low agitation, the dispersion is heated to a temperature of 65° C. to 68° C. until a solution is formed. The solution is then allowed to cool to about 30° C. The solution may then be filtered and placed into a container suitable for use in the lyophilization procedure.

Any conventional lyophilization procedure which would be recognized by those skilled in the art may be utilized to prepare the lyophilized amiodarone formulation. However, it is preferred that the following procedure be followed.

First, the lyophilizer shelves are cooled to a temperature of about 5° C. The amiodarone solutions are placed on freeze dryer shelves and thermocouples are inserted into the containers. The amiodarone solutions are then cooled to a temperature of about −40° C. at the fastest rate possible (preferably about 2 hours). The solutions are held at this temperature for a period of at about six hours. The vacuum in the lyophilizer is then applied at a pressure of about 60-100 M Torr and preferably about 80M Torr. The temperature of the solution is then raised to about 20° C. at the fastest rate possible (preferably about 48 hours). The solutions are held at this temperature for a period of at least 12 hours. At that point, the vacuum may be broken with nitrogen and the containers are stoppered.

The present invention is further directed to methods for treating arrhythmia in a human patient comprising intravenously adminstering to the patient a composition comprising amiodarone in an amount of from 0.9 to 30 mg/ml, lactic acid buffer and water, the solution having a pH of 2.5 to 4.5. Prior to adminstration, the solution may be diluted with 5% dextrose to a concentration of 0.5 to 15 mg/ml of amiodarone.

The following examples are presented to illustrate the production of the formulations of the present invention, rather than to limit the scope of the invention.

### EXAMPLES

### Example 1

A slurry of amiodarone, lactic acid (0.1M) and water sufficient to produce a formulation containing 15 mg/ml of amiodarone was prepared. Lactic acid was added to water and the amiodarone was then added to excess to form a slurry.

The slurry was allowed to rotate for 20 hours on a mixer at 50 rpm. This material was then heated to a temperature of about 60-65° C. and the amiodarone went into solution. The solution was cooled to room temperature and a stable solution was formed.

### Example 2

A slurry was prepared by adding an excess of amiodarone to 10 ml of water at room temperature. The slurry was rotated for 24 hours on a mixer at 50 rpm. The slurry was filtered and assayed. The solution was determined to contain 0.9 mg/ml of amiodarone. A stable solution was formed.

### Example 3

A 30 mg/ml solution of amiodarone was prepared as follows:

In a 600 ml beaker was added 350 ml of water. 4.35 ml of lactic acid (from Sigma-Aldrich) was added to the water to form a solution. A sufficient amount of 0.1N sodium hydroxide was then added to the resulting solution to adjust the pH to 3.69. 12.15 grams of amiodarone was then added to the solution using a lighting mixer. The resulting mixture was heated to a temperature of 67° C. and the amiodarone went into solution. This solution was then cooled to room temperature with a nitrogen blanket. The pH was tested and determined to be 3.65. Sufficient water was added to raise the total volume of the solution to 400 ml. The solution was filtered through a 0.2μ Teflon filter and placed into 5 ml ampoules.

### Example 4

Concentrate Formulation

A concentrated amiodarone formulation may be prepared as follows: 10 grams of amiodarone HCL may be added to a 400 ml container. 190 ml of Water for Injection, USP is added to the amiodarone to form a dispersion. 1.80 grams of lactic acid, USP is then added to the amiodarone/water dispersion. With constant stirring, the dispersion is heated to a temperature of 65-68° C. until a clear solution is formed. 13.3 grams of simethicone is then added to the solution. Sufficient water is then added to increase the volume of the solution to 200 ml. The solution is allowed to cool to 30° C. and filtered through a 0.2μ Teflon filter.

### Example 5

Lyophilized Formulation

20 grams of lactose was added to a 400 ml container. 10 grams of amiodarone HCL was then added to the lactose. 190 ml of Water for Injection, USP was added to the lactose/amiodarone mixture to form a dispersion. 1.80 grams of lactic acid USP was added to the dispersion. With constant stirring, the dispersion was heated to a temperature of 65.0 to 68.0° C. until a clear solution was formed. 13.3 mg of simethicone was then added to the solution. Sufficient water was added to increase the volume of the solution to 200 ml. The solution was allowed to cool to 30° C. and was filtered through a 0.2μ Teflon filter. The solution was then placed into 3.0 ml vials and placed into an Edwards lyophilizer. The lyophilization cycle was 60 hours long.

### Example 6

The stability of the lyophilized formulation prepared in accordance with Example 5 was determined as follows. The vials containing the lyophilized cake formulations were placed in a stability chamber and held at three different temperature conditions; 5° C., 25° C. and 40° C. The formulations were also tested for light stability by being maintained at 25° C. under 500 ft. candles. The formulations were assayed at various time points and assayed via HPLC assay for potency. The assay results are set forth in Table 1 below.

| | | Assay | Relateds | Percent |
|---|---|---|---|---|
| Condition | Time | (mg) | (%) | Remaining |
| 5° C. | 0 | 152 | 0.09 | 100 |
| | 2 w | 152 | 0.11 | 100 |
| | 4 w | 149 | 0.12 | 98.3 |
| | 8 w | 150 | 0.09 | 98.68 |
| | 12 w | 151 | 0.12 | 99.34 |
| | 26 w | 152 | 0.09 | 100 |
| | 39 w | 151 | 0.11 | 99.34 |
| | 52 w | 150 | 0.13 | 98.68 |
| 25° C. | 0 | 152 | 0.09 | 100 |
| | 2 w | 150 | 0.12 | 98.68 |
| | 4 w | 150 | 0.14 | 98.68 |
| | 8 w | 150 | 0.11 | 98.68 |
| | 12 w | 151 | 0.11 | 99.34 |
| | 26 w | 151 | 0.07 | 99.34 |
| | 39 w | 151 | 0.10 | 99.34 |
| | 52 w | 150 | 0.10 | 98.68 |
| 40° C. | 0 | 152 | 0.09 | 100 |
| | 2 w | 150 | 0.14 | 98.68 |
| | 4 w | 150 | 0.14 | 98.68 |
| | 8 w | 152 | 0.10 | 100 |
| | 12 w | 152 | 0.19 | 100 |
| | 26 w | 151 | 0.21 | 99.34 |
| | 39 w | 149 | 0.22 | 98.03 |
| | 52 w | 150 | 0.28 | 98.68 |
| Light | 0 | 152 | 0.09 | 100 |
| | 2 w | 148 | 0.23 | 97.37 |
| | 4 w | 147 | 0.20 | 96.71 |

The present invention may be embodied in other specific forms without departing from the spirit and essential attributes thereof and accordingly, reference should be made to the appended claims, rather than to the foregoing specification as indicating the scope of the invention.

1. A parenteral solution suitable for intravenous administration comprising amiodarone in an amount of from 0.9 mg/ml to 30 mg/ml, a lactic acid buffer and water, the solution having a pH of 2.5 to 4.5.

2. A parenteral solution as in claim 1, wherein the solution has a pH of 3.5 to 3.8.

3. A parenteral solution for intravenous administration comprising, amiodarone, in a concentration range from 0.9 to 10 mg/ml and lactic acid, the solution having a pH within the range of 2.5 to 4.5.

4. A parenteral solution as in claim 2, wherein the solution has a pH of 3.5 to 3.8.

5. A parenteral solution as in claim 1, comprising amiodarone in an amount of from 0.9 mg/ml to 15 mg/ml.

6. A parenteral solution as in claim 5, comprising 15 mg/ml of amiodarone.

7. A lyophilized formulation of amiodarone comprising amiodarone in an amount of from 10 mg/ml to 50 mg/ml, a lactic acid buffer and lactose.

8. A lyophilized formulation as in claim 7, further comprising simethicone.

9. A method for treating arrhythmia in a human patient comprising intravenously administering to said patient an effective amount of a solution comprising amiodarone in an amount of from 0.9 mg/ml to 30 mg/ml, a lactic acid buffer and water, the solution having a pH of 2.5 to 4.5.

10. A method for treating arrhythmia in a human patient comprising intravenously administering to said patient a solution comprising 0.5 mg/ml to 15 mg/ml of amiodarone, a lactic acid buffer and water.
